(19) United States
(12) Patent Application Publication
(10) Pub. No.: US 2013/0218060 A1
(43) Pub. Date: Aug. 22, 2013

(54) Adhesive Wrist Support System

(71) Applicant: Donald P. Bushby, Houston, TX (US)

(72) Inventor: Donald P. Bushby, Houston, TX (US)

(21) Appl. No.: 12/906,054

(22) Filed: Oct. 15, 2010

Related U.S. Application Data
(60) Provisional application No. 61/279,298, filed on Oct. 19, 2009.
(60) Provisional application No. 61/318,602, filed on Mar. 29, 2010.

(51) Int. Cl. A61F 5/01

(52) U.S. Cl. CPC A61F 5/013
USPC 602/21

(57) ABSTRACT

A support system for treatment of repetitive stress injuries. The support system uses a support layer formed from a thin, flexible, stretch resistant material. An adhesive is used that has a high shear strength to prevent the support layer from slipping when applied to the patient. The adhesive loses considerable adhesion as temperatures approach 125 degrees Fahrenheit. This allows the support system to be easily removed at temperatures within the comfort range of the user by soaking in hot water or use of a hair dryer. The system is applied to a wrist or other injury by securing around the wrist. A second portion of the support layer extends up to a portion of the back of the wrist. The user is able to move and flex the wrist through the full range of motion, however with a dampening effect which promotes neutral wrist postures and minimizes accumulative irridation to the median nerve.

Related Applications: This application claims the benefit of provisional patent applications 61/279,298, filed on Oct. 18, 2009 and Ser. No. 61/318,602 filed on Mar. 29, 2010

### FIELD OF THE INVENTION

The present invention relates generally to wrist supports and particularly, but not by way of limitation, to an adhesive wrist support for prevention or treatment of Repetitive Motion Injuries (“RMI”) such as Carpal Tunnel Syndrome (“CTS”).

### BACKGROUND OF THE INVENTION

Carpal tunnel syndrome (“CTS”) is a medical condition in which pain, numbness or a tingling sensation is felt in the area of the wrist, thumb and fingers. The condition may become quite painful, debilitating, thus limiting activities, and in the extreme case leading to corrective surgery. CTS is classified as a cumulative trauma disorder, an ailment that attacks the body's musculoskeletal system. The musculoskeletal system is a system made up of muscles that pull on tendons and move the bones at joints. This condition may be better understood by a discussion of the anatomy of the hand.

FIG. 1a is a dissected (palm side) view of a human hand and wrist (1) provided to illustrate some of the key anatomical features of the hand and wrist. The median nerve (3) runs laterally from the arm, through the wrist (2) to the thumb (5) and fingers. Nerve branches (8) extend from the median nerve to the thumb (5), index and forefingers. The flexor retinaculum ligament (4) bridges between the bones of the hand and forms the roof of and aperture known at the carpal tunnel. Tendons (9) are surrounded with a tendon sheath (9A) and share space in the carpal tunnel with the median nerve (3). The tendons (9) move with every movement of the fingers or bending of the wrist. The cumulative degree of movement of the tendons is proportional to the degree and frequency of movement in the fingers and/or wrist. The tendon sheaths (9A) lubricate and insulate the tendons from direct contact with other tendons, nerves or other soft tissues. Steep wrist angles increase the side loading and the friction caused by tendon movement.

FIG. 1b illustrates an internal view of a cross section of the palm of the hand taken along Section A-A of FIG. 1a. The aperture formed by the flexor retinaculum (4) and bones of the palm section of the hand (18) is called the Carpal Tunnel (10). The carpal tunnel (10) is a conduit for the median nerve (3), and tendons (9). The tendons are covered with a thin protective “tendon” sheath (9A).

FIG. 2 illustrates a dissected (palm side) view of the hand-wrist area, from the mid-palm to the lower portion of the forearm. The bones of the arm are illustrated by items (12) and (13). The median nerve (3) is illustrated running under the palmar carpal ligament, under the flexor retinaculum (4) (i.e. known as the roof of the carpal tunnel) toward the thumb (5) and fingers, via the carpal tunnel (10). The nerves (8) leading toward the thumb and fingers are illustrated as well as, are the bones of the wrist (16). The outer skin layer (14) in indicated at a point in the forearm region of the arm. The wrist area (15) joins the arm and hand.

Carpal Tunnel Syndrome (“CTS”) results from inflammation of the median nerve in the region where it passes through an aperture bounded by the bones of the wrist (carpal bones) and the flexor retinaculum. This aperture is commonly referred to as the carpal tunnel. The median nerve supplies sensation to the wrist area as well as the thumb, the index finger, the middle finger and the ring finger. Therefore, when the median nerve becomes irritated, pinched or inflamed in the carpal tunnel area, the aforementioned sensations are felt in these areas of the hand and wrist. Carpal tunnel syndrome specifically affects the sensitive nerves of, including the median nerve and the blood supply that feeds, the hands and wrists. Restrictions in blood flow can create higher than normal pressures in the hand, resulting in impinging forces applied to the median nerve. Conversely, inflammation in the carpal tunnel area can also result in blood flow restriction to the hand.

Inflammation in the median nerve can be the result of repetitive flexations of the wrist. These repetitive movements are required in numerous activities such as operating a computer keyboard, cash register, using power tools, or holding a steering wheel. CTS is referred to sometimes as a Repetitive Motion Injury (“RMI”). One treatment for the development of CTS, and preventing aggravation of an already existing CTS condition is the limitation of the range of motion and flexibility of the wrist, by means of an external support or brace. Lessening the repetitive flexations of the wrist thereby lessens the frictional forces applied to the median nerve and reduces the likely of the condition becoming inflamed or developing. Dampening the motion of the wrist and maintaining good wrist posture (i.e. neutral alignment) lessens the cumulative irritation of the nerves and tendons in the carpal tunnel.

A number of treatments and approaches are used to treat CTS, including the use of rigid splints, braces and/or elastic and compressive supports. Wrist braces, splints and supports are often used to immobilize the wrist, in an attempt to reduce wrist flexure and/or maintain alignment. Over the counter non-inflammatory medicines are often used. Steroid injections are often prescribed, and corrective surgery is used in more extreme cases. Steroid or non-inflammatory injections are often painful, and they risk the introduction of infection, degradation of tissues, or rupture of a tendon. Carpal release surgery is expensive and is not always successful. Recovery time from such surgery may range from days to weeks, during which time the patient has to curtail activities. Even with the available conservative treatments on the market, as many as 260,000 corrective carpal tunnel release surgeries are performed each year.

Most wrist supports in general and particularly those for prevention of carpal tunnel syndrome consist of either a substantially elastic or substantially rigid materials or a combination of both, secured about a person's wrist. Typically, such wrist supports consist of strips of material which, when applied (i.e. usually encircling) to a person's wrist, support the wrist and limit its flexibility. These wrist support typically encircle the entire wrist and often include elastic materials which may apply a compressive force to the hand/wrist area and/or include rigid elements to immobilize or restrict flexation of the hand to wrist area. The rigid supports and braces immobilize the wrist to varying degrees using rigid materials of various compositions, shapes and sizes. The rigid materials often utilized in these braces are metal(s) or thermoformed plastics. The softer materials include an array of fabrics, or cushioning materials, usually sew or otherwise adhered together. These devices use a wide variety of fasteners, the fasteners can range from hook and loop fasteners, hooks, loops pull strings etc.

Alternatively, very lightweight adhesive strips are available in the market, which provide bio-feed back to the user, regarding the position of the wrist with regards to the angle of flexation or of rotation of the wrist with regards to the forearm. [These contact the wrist on a single side only, typically top or bottom (i.e. palm side) and provide minimal support to the wrist. Alternatively, two narrow strips are applied to the wrist, one on the top and one to the bottom of the wrist-hand-forearm to provide biofeedback (particularly during sleep). Another product that is sometimes used for treatment includes Kineso Tape distributed by Kineso USA which is an elastic tape that is supposed to promote circulation without restricting movement. Strapping techniques are well known and are also well known. These usually use a two step application process of first applying a cohesive, elastic layer, and then wrapping in an overlapping manner a plurality of strips or windings about the body part. These are hot, difficult to apply, apply compression to the medial nerve area, do not extent typically to the back of the hand, do not have the structural support advantage of direct skin contact and are not pre-shaped or pre-formed for the application, thus resulting in lack of convenience.

Most conventional wrist braces of the type discussed above are bulky, and not often of suitable or desired appearance for use in an office or professional environment. Many of these interfere with normal use of the hand and/or fingers, interfere with the sleeves of shirts, or blouses and cannot be discretely worn by the user. When rigid materials are used they are often bulky, uncomfortable, and irritating against the skin. Heavy or cushioning materials or layers used restrict heat transfer away from the body, resulting in the user feeling hot or uncomfortable.

A further disadvantage of support devices which encircles the wrist, is that they often apply a compressive force to the wrist/hand area. This compressive force acts in the direction of closing or restricting cross sectional area of the carpal tunnel, often by pushing inward on the roof of the tunnel structure. Restricting the cross sectional area of the carpal tunnel can increase pressure on, and increase the likely hood of irritation of, the median nerve.

A further disadvantage of the prior mentioned adhesively applied products is the pain and discomfort caused during removal. Users are unlikely to remain compliant with using a support device which causes pain every time it is to be removed, by pulling the hair or aggravating the skin surface.

Clearly, a need exists for improved preventative measures and treatments that are simple to use, non-invasive, cost effective and which therefore encourage use and increase treatment compliance.

### BRIEF SUMMARY OF THE INVENTION

A preferred embodiment of the present invention provides a wrist support, which is light weight, economical and may be easily applied by the end user. It also provides a wrist support system, which is adhesively applied directly to the skin of a user to stabilize the wrist and which may be used in the prevention of or treatment of CTS. The wrist support utilizes a durable support layer which in a preferred embodiment is substantially resistant to elongation. In a preferred embodiment includes a heat sensitive adhesive and method of removal which does not cause pain or discomfort to the user.

A preferred embodiment of the present invention provides a wrist support that does not interfere with use of the fingers or thumbs and may be used during typical repetitive motion activities such as working on the computer, playing a guitar or preparing food. The invention dampens normal movement of the wrist without impairing the normal range of motion. Irritation of the median nerve is lessened as the user reduces the angular degree and frequency at which the hand is moved relative to a neutral wrist posture via use of the device. This reduced angular degree of normal movement of the hand from a neutral wrist position along with the reduced frequency of such movements lessens the degree of ligament movement in the carpal tunnel. Lessening ligament movements and lowering ligament sidewall stress resulting from steep wrist angles lowers the cumulative irritation to the medial nerve. This reduction of irritation to the median nerve is therapeutic in preventing, treating and relieving symptoms of carpal tunnel syndrome.

The preferred embodiments of the present invention is light weight, can be discretely worn and installation or use of the device does not require detrimental compressive forces be applied to carpal tunnel structure. The application of external compressive forces on the carpal tunnel increases sidewall pressure internal to the carpal ligaments, thus increasing likelihood for nerve irritation to develop as the ligaments move with respect to the median nerve. The present invention performs wrist stabilization and provides anatomical support of the hand and wrist interface region without requiring or applying compressive forces to the carpal tunnel.

The present invention in a preferred embodiment provides a wrist support that is effective, yet comfortable to apply, to use and to remove. Compliance with any treatment modality is essential to its effectiveness. The wrist support of this embodiment is light weight, economical, and disposable (i.e. intended for a single use followed by being discard rather than for multiple re-use). The device is typically pre-shaped and provided to the user in a ready to apply condition. The device can be applied without requiring use of scissors, other tools and does not require special skills or training. Since the device is easy to apply and can be discretely worn, users are more prone to use the device over more cumbersome or bulky, visually intrusive wrist support devices. The device is intended for a single time use followed by disposal, thus improving the level of sanitation of the device over devices which are used multiple times. The aforementioned advantages aid in compliance by end users with use of this invention. Consistent use of the device over a period of time (e.g. a number of days or weeks) is believed to produce the highest therapeutic benefits and reduction of pain and/or CTS symptoms.

An embodiment of the invention reveals a wrist support system that is adhesively applied to the skin of the user. The wrist support may be a thin one-piece device made of a stretch resistant material of a uniform thickness. Alternatively, the wrist support may be made with a stretch resistant material bounded by less uniform and more deformable material. The wrist support may be also be of a multi-piece design with each piece applied individually. The wrist support may be shaped for ease of conformance to the wrist area of the human body. The support layer has an adhesive layer for adhering the support layer to the outer skin tissue of the body. The adhesive layer may cover all or only a portion of the support layer. The adhesive layer is provided with a removable protective cover.

A preferred embodiment utilizes an adhesive layer that is high strength to be effective, yet is able to be easily removed without discomfort. Previous devices used a strong bonding adhesively applied support or splint that had the disadvantage during removal of pulling the hair of the user in the area of application. The present embodiment uses a permeable (i.e. woven material in a preferred embodiment) support layer which allows moisture and moisture vapor transfer readily through the support layer. This feature in combination with an adhesive layer which releases when immersed in warm water, allows the device to be easily removed with out causing discomfort or pulling of hair. A user may apply an adhesively applied orthotic support for a period of time (preferably while performing repetitive motion actives) then remove the support, preferably discarding it. The user can then apply a second support, wear it for a period of time, discard and repeat the process. The reduction in cumulative irritation afforded to the user will in many cases allow symptoms (i.e. pain and numbness to subside) as internal inflammation in the carpal tunnel diminishes. The user is then often able to avoid costly, painful and/or invasive procedures and the associated loss of productivity.

A preferred embodiment provides a wrist support for repetitive stress injuries that does not use compression. Blood flow is essential to the recovery and healing of body tissue from damage. The lack of compressive force around the wrist aids in the maintenance of normal blood flow. During normal movement of the wrist, the outer skin surface down all sides of the wrist is extended and/or compressed as the wrist move from away from (or towards) a neutral wrist position. The differential movement of the outer skin layer due to the tension in the support layer results in a massaging action between the outer dermis and inner structures which enhances blood flow.

The wrist support, in a preferred embodiment, may be shaped to completely encircle the wrist or only partially circle the wrist. For example, the wrist support may cover greater than 50%, 70% or 90% of the circumference of the wrist, thus contacting the wrist on 2, 3 or all 4 sides (i.e. top, bottom/palm, inner and outer sides). The wrist support consists of a support section for adhering to the wrist only and optionally with portions for extending and adhering to the backside of the hand (i.e. across the wrist-hand boundary) and optionally into the palm area. When a portion of the wrist support is used which extends from the wrist, up the back of the hand, this section may extend only partially such as less than 1 inch or it may extend fully up the back of the hand to near the first knuckle. The removable protective covers may include tabs for ease of removal of the protective cover(s) from the support layer. The protective covers may be singular or may have multiple sections to improve the ease at which the wrist support is applied to the body. The wrist support may be pre-shaped for ease of conformance to the portion of the hand-wrist area for it is to be applied. Alternatively the wrist support may be cut, torn or otherwise trimmed by the user to improve conformance.

One embodiment of the present invention include a process for providing support to a wrist. The process includes providing a thin, non-rigid support layer having an adhesive layer and a cover layer. The process includes removing the cover layer from the adhesive and the support layer and applying the support layer directly to the outer skin tissue on portions of the wrist, and/or hand. The process may include adjusting the position of the support layer to the portion of the wrist or to portions of the hand when the applying step results in an unsatisfactory application. The adjustment may be made by way of example to increase or decrease the pre-load/tension on the back of the hand, or to modify the level of proprioceptive awareness provided by the device. To stabilize the wrist symptoms a support layer is adhered to the skin of the user around or at least partially the wrist of the user. The support layer restricts stretching of the skin tissue, thus damping the flexation of the wrist.

One preferred embodiment includes a process for applying a pre-load to the support device. When portions of the wrist support extend to the back of the hand, and are adhered to the back of the hand, downward flexation of the wrist is further dampened, and proprioceptive awareness of the position of the wrist is increased. When a portion of the wrist support is adhered to the back of the hand while the hand is in an upward flexed position a preload or pretension is applied to the wrist support, which is experienced by the hand, increasingly as the wrist angle is decreased (i.e. moved downward) toward a neutral position. The more preload or pretension placed on the wrist support, the greater the damping of the wrist with regards to downward flexations. A single strap around the wrist and adhered to the wrist may be used to provide anatomical stability to the musculoskeletal system of the wrist. The wider the strap, generally the more support provided. Additional, support benefit diminishes as the support gets wider and extends further away from the wrist in the direction of the forearm. When the support layer is applied around the wrist at the fulcrum, pivoting at the fulcrum can be directly damped and/or is at least partially restricted.

An alternative embodiment of the present invention includes an article of manufacture (i.e. sheet of material) for stabilizing the wrist. The article includes a sheet of material including a support layer having a thickness by way of example of less than 25 mils, an adhesive layer, and a release liner. The sheet of material may be provided with shapes already cut-out and ready to apply or these shapes may be cut out by the user. This embodiment is a sheet of material including a support layer, with suitable mechanical properties (e.g. low deformability under cyclic loading, and minimal elongation) and an adhesive layer and a removable cover layer. The support layer may be a single layer of woven or non-woven fabric, which has stretch resistant properties and an adequate adhesive layer to maintain the support system in place on the user for the intended period of use and without unnecessary discomfort while removing it. The support layer of this material may be in the range from about 2 mils to 100 mills in thickness. The support layer may include Rayon or other fibers, natural or synthetic.

The present invention in one preferred embodiment includes an assembly for stabilization of the wrist. The assembly includes a sheet of material configured to be affixed to at least a portion of a the wrist, the sheet including a stretch resistant support layer joined to a cover layer by adhesive, wherein the cover layer is removed from the adhesive and the support layer is affixed to the portion of the wrist-hand region by the adhesive. The assembly includes instructions instructing how to apply the support layer to the wrist and/or wrist-hand region for stabilization of the wrist. The wrist support may include a plurality of separate sections, which are applied separately the body, to provide the degree of support required.

A preferred embodiment of the present invention include a method of manufacture for producing low cost, disposable (i.e. one time use) stabilizing wrist braces. The method includes: A) providing a planar sheet of material of uniform thickness, wherein the planar sheet includes a support layer, and adhesive layer and a cover layer removably disposed over the adhesive layer B) feeding the planar sheet in to a cutting machine, wherein the cutting machine is a die cutter or laser cutter C) and cutting through the support layer to create shapes in the support layer which are for ease of conformance to the wrist or hand-wrist area of the human body. The die cutter may be a rotary or a plate type cutter. The feeding mechanism may be utilized the planar sheet in rolled form and feed it though a series of rollers and tensioning devices. The cutting device may be configured to only cut through the support layer, leaving the protective cover layer intact and thus not cut in a shape matching the support device. The protective cover layer can be handled and packaged in sheets or rolls for ease of dispensing of the adhesive supports. The rollers may be powered providing the motive force to the process. The end product may be rolled for final packaging steps. Alternatively, both the support and cover layers may be cut completely though, forming complete articles which can be immediately separated from the planar sheet after the cutting process.

These and other features of the present invention will be evident from the ensuing detailed description of preferred embodiments, the drawings and from the claims.

### BRIEF DESCRIPTION OF THE DRAWINGS

FIG. 1a is a dissected (palm side) view of a human hand and wrist (1) provided to illustrate some of the key anatomical features of the hand and wrist.

FIG. 1b illustrates an internal view of a cross section of the palm of the hand taken along Section A-A of FIG. 1a to illustrate some of the key anatomical features of the carpal tunnel.

FIG. 2 illustrates a dissected (palm side) view of the hand-wrist area, from the mid-palm to the beginning of the forearm.

FIG. 3 illustrates a perspective view of a preferred embodiment of the invention in place on the wrist and hand of a user.

FIG. 4 illustrates a perspective view of a prior art device on a human hand.

FIG. 5a illustrates a side view of a prior art device on a human hand operating a computer mouse.

FIG. 5b illustrates a palm side view of the same prior art device of FIG. 5a.

FIG. 6 illustrates a perspective view of an embodiment of the current invention, illustrating the invention applied to the back of the hand, and encircling the wrist.

FIG. 7 illustrates a perspective view of the current invention in an embodiment, which does not completely encircle the wrist, and does not apply compression to the median nerve.

FIG. 8 illustrates a perspective view of an embodiment of the invention in a simple form with no extension to the back of the hand and completely encircling the wrist, and overlapping itself.

FIG. 9 illustrates the current invention from palm side view of the hand-wrist area of FIG. 3, this illustrating various degrees of wrist encirclement.

FIG. 10 illustrates a layout of an embodiment of the invention in a universal (i.e. right or left wrist) format, with an elongated wrist portion and a portion for extending up the back of a hand.

FIG. 11 illustrates a layout of an embodiment of the invention with and elongated wrist portion and a portion for extending up the back of the hand.

FIG. 12 illustrates another embodiment of the invention.

FIG. 13 illustrates the support, adhesive, and removable cover layer of the invention.

FIG. 14 illustrates the current invention in a kit form including packaging, instructions and the support material.

FIG. 15 illustrates an efficient process for manufacturing anatomical supports for the body, particularly for the wrist.

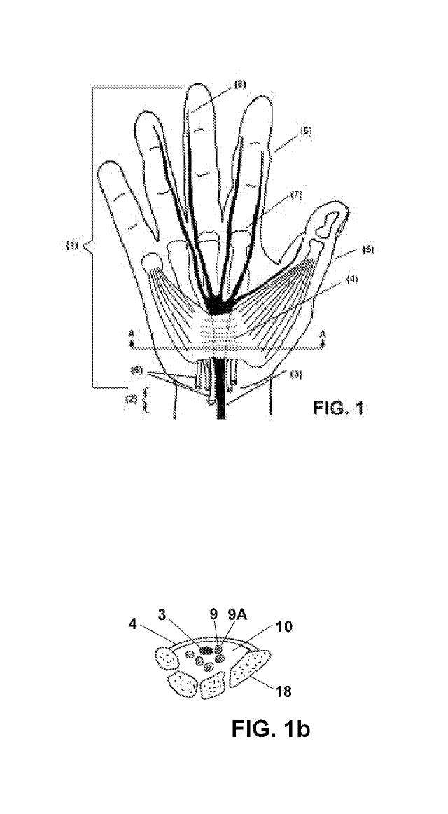

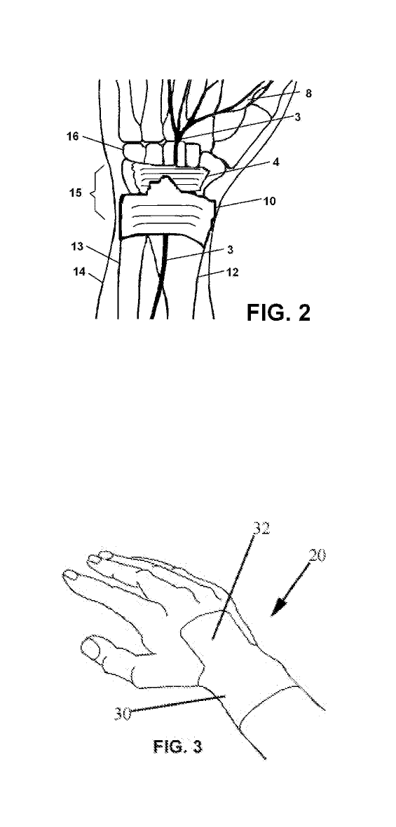

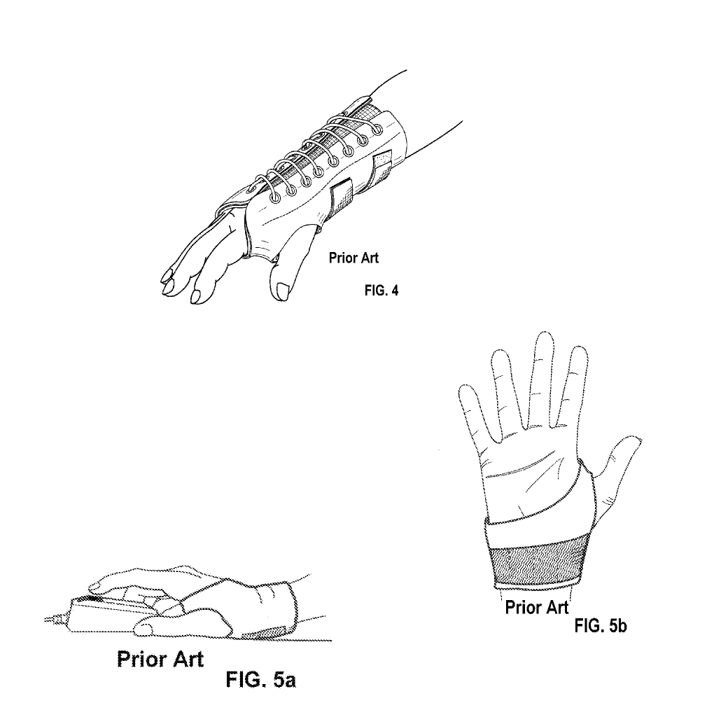

### DETAILED DESCRIPTION OF PREFERRED EMBODIMENTS

The present invention provides a disposable prefabricated orthosis device for support of the wrist, the treatment of carpal tunnel syndrome as well as other wrist issues. Descriptive embodiments are provided before for explanatory purposes. It is to be expressly understood that the present invention is not be limited to these descriptive embodiments. Other embodiments are considered to be within the scope of the present invention, including without limitation the use of the present invention for other applications, such as ankle supports and supports for other body parts.

### Definitions

The following definitions are provided for explanatory purposes.

A prefabricated orthosis is any orthosis manufactured in quantity without a specific individual in mind (e.g., off-the-shelf). Prefabricated orthotic devices may include custom-fitted devices (e.g., trimmed, bent or molded for use by a specific individual).

A custom-fabricated orthosis is one that is specifically manufactured for an individual. Custom-fabricated devices may include custom-molded devices (e.g., molded to the individual's specific body part).

Limb orthoses typically fall into the following categories: rigid and semi-rigid; custom-fabricated orthoses; semi-rigid prefabricated and flexible orthoses. Note both rigid and semi-rigid restrict range of motion in at least one direction. The present device dampens movements while allowing full range of motion.

Disposable or Single-Use means designed to be disposed of after a single application or use. These are not intended for re-use or multiple applications.

Woven fabrics are defined as fabrics that are formed from interlacing threads or strips angularly to one another.

PSTC refers to standards provided by the Pressure Sensitive Tape Council, an industry group.

Non-sterile—not sterilized or design for use over wounds or in wound care.

### Overview

A preferred embodiment of a wrist support according to the present invention includes a thin flexible, inelastic sheet member (i.e. support layer) of preferably substantially uniform thickness The wrist support also includes a tack adhesive layer provided on at least a portion of a surface of inelastic support layer to secure the support device on the hand and wrist. A releasable cover layer is used to cover the adhesive layer until the device is prepared for use. The device in a preferred embodiment is non-sterile and not intended for use in wound care.

### Support Layer

The support layer is formed from woven materials, although non-woven materials may be used in some embodiments. This support layer should be preferably non-rigid, conforming easily to the contour of the human body. The inelastic support layers may be any desired synthetic or natural materials. A support layer thickness of less than 100 mils, preferably less than 30 mils and, with particular preference, less than 5 mils is desired. Materials of much greater thickness could be used, however, thicker materials have a tendency to conform less easily to the contours of the body, than thinner material of the same composition. A support layer which readily conforms to the three-dimensional contours of the hand and wrist area is desired, wherein increased adhesion as a greater effective contact surface area is provided. The support layer may be a composed of a single layer or a plurality of layers of materials. Semi-rigid materials could be used in conjunction with the thin flexible material, for example a thin plastic strip may be used across the wrist joint or on the back side of the hand. This more rigid yet flexible material may be used to provide support in addition to the support provided by the support and adhesive layers alone.

The support layer of this embodiment is substantially inelastic. While this embodiment is substantially inelastic, elastic or partially elastic materials may be used for the support layer for applications requiring a low level of support. The support layer should have adequate tensile strength for the application. The ultimate yield strength for the support layer of at least 25 N/cm is preferred, most preferably from 30 to 150 N/cm and, with particular preference, from 50 to 125 N/cm.

The preferred material is a woven micro-fiber with high strength fibers. The thinness, strength, breathability and high compliance/ease of conformance to the body makes micro-fiber nearly ideal for the application. The preferred woven micro-fiber is highly permeable, has a high tensile strength and exhibits minimal elongation under load. The material of a preferred embodiment is a uniformly woven fabric which exhibits equal resistance to stretch in a crosswise or lengthwise manner with respect to the weave of the threads. One preferred support layer is a single layer of woven synthetic micro-fiber. The low elongation of synthetic micro-fiber aid it in staying well adhered to the human body, as any elongation in the support layer stresses the adhesive layer along the edge of material deformation leading to loss of bond.

The wrist support can be manufactured and sold at low cost. The wrist brace can be manufacture in a wide range of colors including a wide range of skin tone colors. The thin adhesively applied invention especially when worn in a color close to that of the user can be very discretely worn. The ultimate tensile stress elongation (i.e., at any point of breakage of the support layer is preferably less than 5%-20%, preferably less than 5%. The preferred embodiment of the support layer does not substantially deform under cyclic loading by less than 15% after 25 cycles at a load of 25 N/cm, and preferably less than 5% after 25 cycles at a load of 25 N/cm. The preferred embodiment of the support layer also has an elongation of less than 15% at a load of 10 N/cm is preferred, with particular preference to an elongation of less than 5%. One preferred embodiment has an elongation of less than 1% at a load of 10 N/cm.

The support layer in a preferred embodiment may be hand tearable, although other embodiments need not be hand-tearable. The hand terability improves ease of adaptability of the device by the user to account for variations in wrist/hand size or installation preference. The edges should be arced, rounded or tapered to prevent catching on clothes or other objects.

The wrist brace can be manufacture in a wide range of colors including a wide range of skin tone colors. The thin disposable adhesively applied invention especially when worn in a color close to that of the user can be very discretely worn.

The support layer is preferably vapor and water permeable to allow the skin to breathe during use. This improves the comfort to the user and increase the compliance of use.

### Adhesive

The tack adhesive layer may include any tack adhesive suitable for temporarily attaching the inelastic support layer to wrist-hand area of the body. Preferably, the tack adhesive used in the tack adhesive layer is sticky enough to reliably hold the wrist brace in place in its installed position without substantial slippage, but is not so sticky that it causes difficulty or discomfort in removing the wrist brace from the skin tissue on the wrist or hand. The adhesive layer is preferably hypoallergenic, and by way of example latex-free. Adhesive layers with high sheer strength characteristics are preferred. Preferably the adhesive has a sheer strength of at least 15 oz/in under the PSTC-3 standard. The adhesive layer may be, in a preferred embodiment, quite thin in order to minimize slippage under sheer stress. Adhesive layers less than 5 mils thick are preferred, and particularly a thickness of less than 3 mils. The preferred adhesive layer will allow the wrist support to be removed with minimal pain or discomfort.

The adhesive of a preferred embodiment of the present invention has a high shear strength so that it does not easily loosen during normal activities. The adhesive of a preferred embodiment does easily release when exposed to a temperature of about 125 degrees Fahrenheit or more. Examples of pressure sensitive adhesives that lose their adhesive strength at temperatures above ambient temperatures is the MACtac Incorporated MP-894 and TM8946 by MACtac North America Incorporated. It is to be clearly understood that other adhesives may be used as well. This embodiment uses a support layer that is permeable so that patient can simply place the hand and wrist area beneath running hot water to release the adhesive so that the support device is easily removed without damage or discomfort to the patients skin. A hair dryer could also be used to release the adhesive.

The adhesive of a preferred embodiment has an adhesion strength which decreases in adhesion by at least 20% when its temperature is increased from 70 deg F. to a steady state temperature of 110 deg F. whereby minimizing discomfort to the user during removal by the application of heat. This allows the support layer to be easily removed.

Characteristics of the adhesive preferably include without limitation:
Non-skin-irritating pressure sensitive adhesive, with good shear resistance, good tack and medium peel adhesion.
Dermatological non-irritability, suited for direct skin contact, preferably latex free and hypoallergenic.
The adhesive preferably will be selected would lose >greater than 50% of its adhesive ability in either shear or peal strength when expose to heat (100-125° F.) for by way of example 100° F. for 30 seconds or 125° F. for less than 20 seconds, thus allowing easy removal with the aid common tap water in the 100° F. to 125° F. range.
Shear adhesion: per (PSTC 7); Preferred >150 g/0.25 inch2, more preferred >250 g/0.25 inch2, most preferred >400 g/0.25 inch2 when test at 74° F. for 24 hours. Each of the above having a shear adhesion failure temperature of lower than 160° F., more preferably lower than 150° F., and most preferably between 130-140° F., when tested at a value in X g/inch2 (X=to their corresponding shear adhesion strength as measured above (PSTC 7)).

The higher adhesion levels by way of example >400 g/0.25 inch2 (per PSTC 7) when paired with a strong stretch resistant (i.e. materials which exhibit low elongation under the conditions of the application) support lay by way of example a woven-microfiber can be used in combination to produce a wrist support for suitable for rigorous applications such as used during sporting events, but also light-weight, discrete and comfortable to were in an office environment. The use of woven materials is of particular benefit at it allows warm-hot water to penetrate quickly to the adhesive layer to aid in removal.

The adhesion level preferably will reduce such that removal is not painful to the user. For example: Soaking in warm-hot water (105-125 degrees F.) for 10-30 seconds will aid in removal, particularly when the support layer is readily permeable (such as a woven material), and the shear adhesion fail temperature of the adhesive is less than 160 degrees F.

### Protective Cover

The protective cover over the adhesive may be a KRAFT trademark paper or other suitable materials. The protective cover may be coated by materials such as silicon to improve the removability of the protective cover from the adhesive layer. The protective covers may protrude beyond the support layer or have tabs for ease of removal of the protective cover from the adhesive. The protective layer may be slit or divided in to distinct separate portions to improve ease of handling during application. A plurality of protective covers aids the user, in applying the device in a prescribed manner, without the adhesive layer adhering to itself. A protective cover layer thickness of less than 40 mils thickness, preferably less than 15 mils, and with particular preference of 5-10 mils is desired. The protective cover layer may have printing, other indications or instructions printed on it for advertisement, warning or general instructions.

### Sizes and Shapes

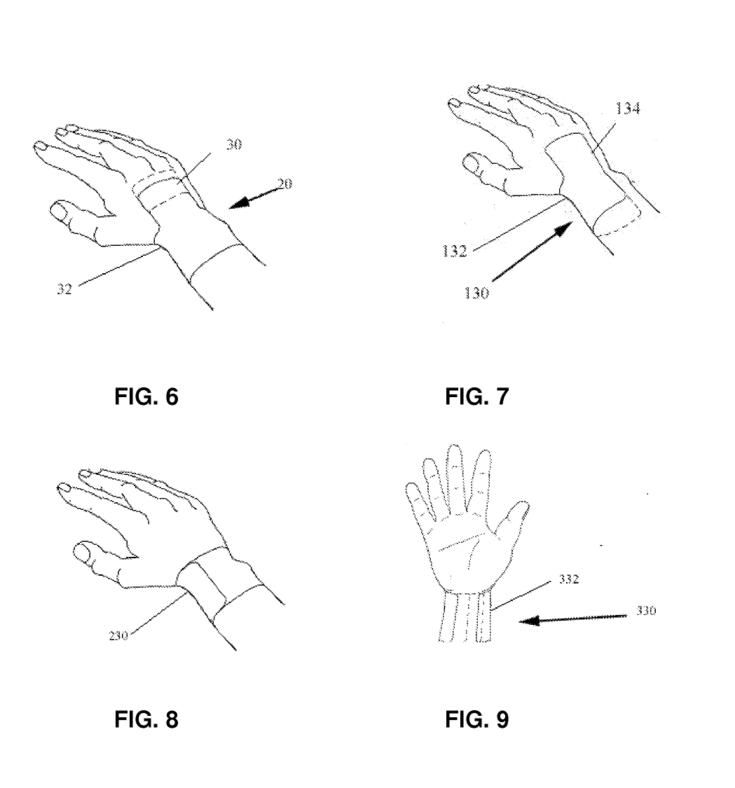

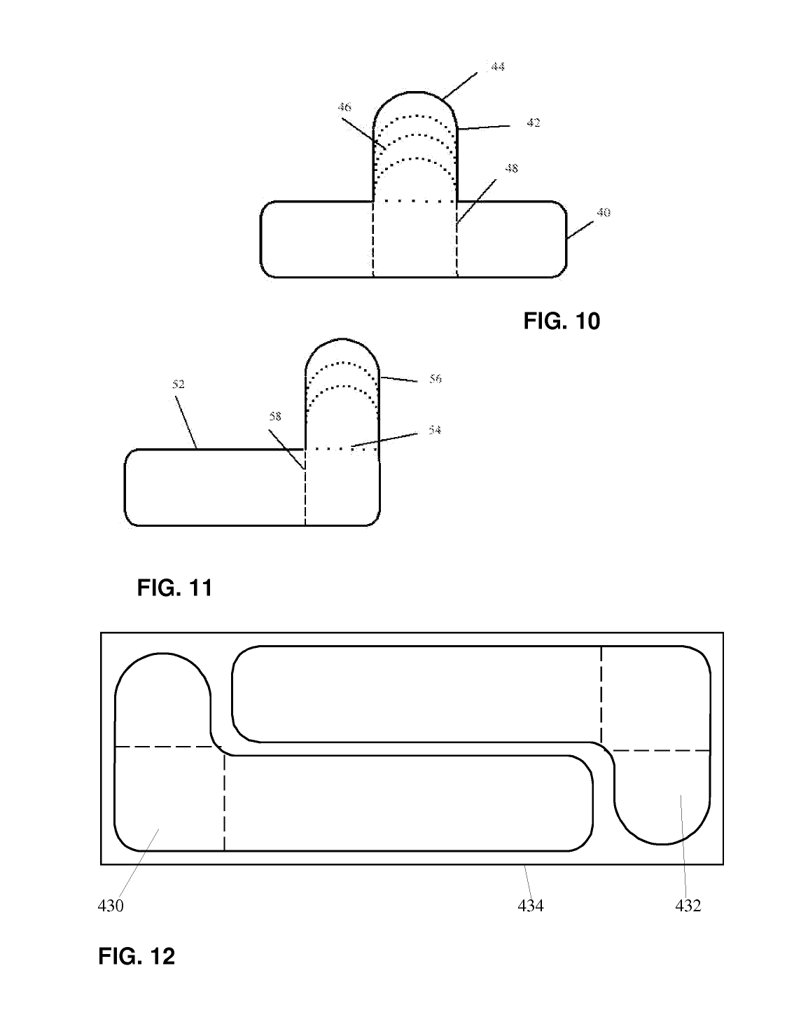

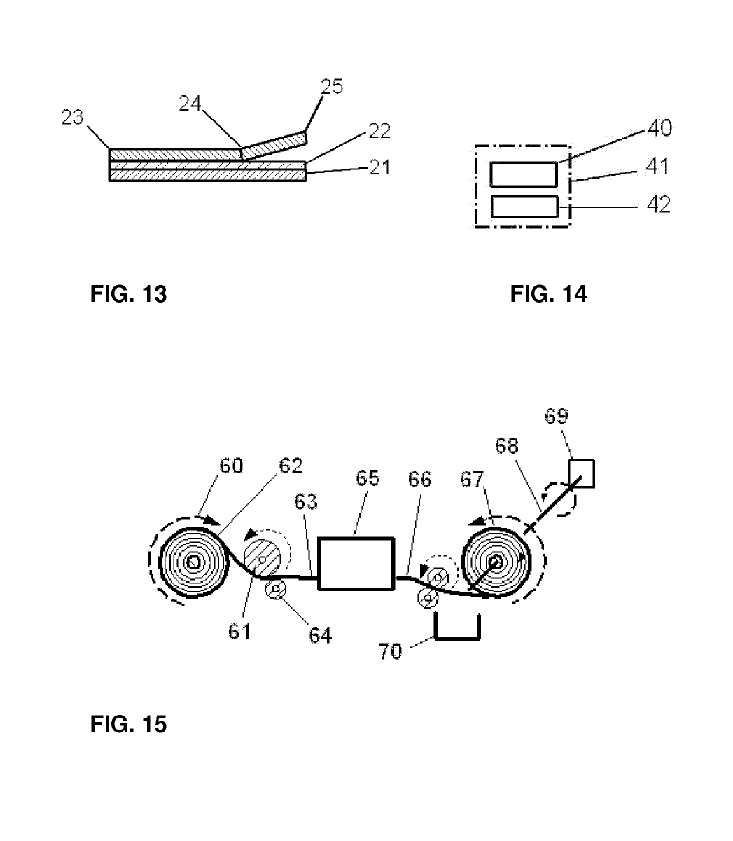

The wrist brace can be of various sizes and shapes each having differing advantages in either degree of support provided, ease of application and efficiency in the manufacturing process. The preferred embodiment of a wrist support 20 of the present invention includes a first portion 30 (as shown in FIGS. 6-11) that encircles the wrist. This portion can completely encircle the wrist but preferably only encircles a portion of the wrist. This minimizes compression on the carpal tunnel region of the wrist which can lead to inflammation of that region worsening the condition. The second portion 32 on the wrist support engages a portion on the back of the hand. The second portion 32 preferably extends from the wrist portion 30 to a point preferably no higher than just below the first knuckle on the back of the hand. Thus, the wrist support will not interfere with the use of the fingers of the patient. The wrist support in one embodiment can be in a substantially T shape, as shown in FIG. 10, or in a substantially L shape as shown in FIG. 11.

Wrist support 20, in one embodiment is illustrated in FIG. 6 applied to the back of the hand portion. Extended portion 32 may extend up to knuckle area of the back of the hand. This portion 32 is shorter in length only partially extending up the length of the hand. The wrist support of this embodiment includes rounded corners to minimize catching on items and pulling loose from the skin although square corners may be used as well. Portion 30 includes an elongated wrist portion, which encircles the wrist at least partially. The edges and corners of this portion may be geometrically shaped (e.g. rounded) to prevent unintentionally pulling away from the skin.

The wrist support 130 of another embodiment, as illustrated in FIGS. 7 and 11 is “L” shaped with the “L” being made up of the intersection of two elongated sections 132, 134. The shorter 134 of these two sections extends up the back of the hand. This portion is adhered to the back of the hand to serve the important role of providing stability to the hand-wrist interface in both major planes (i.e. up/down flexion and side to side flexion). The preferred length of this elongated section is less than about 6.5 inches with a more preferred length of less than 5 inches. The preferred width of this section is less than 3 inches, or more preferred less than 2.5 inches wide, with a preferred minimum width of about 1.5 inches. The length of the longer of the two elongated sections is preferred to be long enough for this section to completely encircle the wrist (typically approximately 8-to approximately 11 inches) and overlap and adhere to itself. Shorter lengths which only partially encircle the wrist are acceptable in some cases. This section should typically adhere to at least three sides of the outer wrist surface in order to improve its effectiveness in providing neutral wrist postures along all major axis of rotation.

Another embodiment of the wrist support 230, as shown in FIG. 8, is an elongated member for support of the wrist, illustrated completely encircling the wrist and adhering to itself (i.e. overlapping). This version of the invention may be used for example in the prevention of impact injuries, during sporting events. The wrist portion can vary in width depending on the degree of support required. Overlapping improves adhesion and support provided by the device.

The wrist support 330 of another embodiment, illustrated in FIG. 9 has an elongated portion 332 for support of the wrist that encircles the wrist or, in some instances only partially encircled the wrist. In the most preferred embodiment the wrist is encircle 360 degrees or greater. The wrist support may also encircle less than the entire wrist.

The wrist support in one preferred embodiment is illustrated in FIG. 10. This embodiment is a pre-cut or pre-shaped article (i.e. wrist support) formed for ease of conformance to the body and thus ease of application by the user. The elongated wrist member 40 with an extension 42 for adhering to the back of the hand splits in the protective cover layer 44 with an optional length of extension for the back of the hand area. The extension 42 can be trimmed along perforations 46. This design can easily be used of either a right or left extremity (i.e. hand, wrist) by trimming along perforations 48. The article can be trimmed or adapted further by the user by hand or with the use of cutting tools if required. Dimensions of the two intersecting elongated member which make up this embodiment would be similar to those found in the “L” shaped device.

Another embodiment of the wrist support is illustrated in FIG. 11. This wrist support 50 has an elongated wrist portion 52 with an extension 54 for adhering to the back of the hand or palm side of the hand. This extension 54 is not centered but extends from one end. Alternatively two extensions could be provided one for the palm side and one for the back-side of the hand. Splits 58 in the protective cover layer 56 and optional length of extension in to the back of the hand area illustrated. This design can easily be used of either a right or left extremity (i.e. hand, wrist) as provided or can easily produced in minor image with the extension on the opposite side.

Another embodiment of the wrist brace is illustrated in FIG. 12. This embodiment uses two L shaped devices 430, 432 provided on a single support layer 434. The user simply selects one device, pulls it from the support layer, trims it to the appropriate size and applies it to the wrist.

Another embodiment of the wrist brace uses a longer extension. The longer extension allows the extension to be applied beyond the knuckles and wrapped around a finger. This provides additional securement to minimize the device from peeling away from the back of the hand or wrist.

The wrist brace of the invention can be provided in kit form and may comprise of a single brace or a plurality of braces in a package. However, due to the inexpensive nature and disposability (i.e. single use nature) of the wrist brace, it may come in a large volume package or dispenser. A large dispenser may be used by way of example: in an office environment. Also, while the package may come in a variety of specific sizes, it can also be provided in a reduced number of sizes, with the ability to be “cut to fit” by the wearer prior to use. The package may include instructions for use, application or modification of the device by the user.

### Manufacture

FIG. 15 illustrates a manufacturing process of producing pre-shaped support devices for the human body. These support devices may be shaped for ease of conformance to the wrist by way of example as found in FIG. 10 and FIG. 11. The process includes a supply of the material which includes a support layer (21), an adhesive layer (22) and a protective cover layer (23). One step includes supplying the material (62) into a cutting machine (65), wherein the cutting machine is a laser cutter or die-cutter. The die cutter may be either a plate cutter or a rotary type device. The material may be supplied in roll form (62) and powered though a series of guide rollers (61) and tensioning elements (64) by a motive device (69) such as an electric or hydraulically driven motor. The motive device (69) may transmit it motive (rotary) force to one or more of the guide rollers (61) or to the post-cut reel (67). Motion (rotary) as a result of the applied motive force is illustrated by a series of arrows, (60) illustrates the motion of the supply reel. The rollers feed the (65) cutting machine with a sheet of material (63) of adequate speed and tension for proper cutting operation. A power linkage (68) is used to transmit motive force from the motive device (69). The step of cutting includes cutting the material of FIG. 8 to a predetermined pattern or shape. The cutting step may cut all layers (21) (22) & (23) allowing the device to be removed from the larger sheet of material. Alternatively, only the support layer (21) will be cut allowing the device to remain with the larger sheet of material as it moves into the final section of the machine. In cases where all three layers are cut the devices may be separated immediately after the cutting step, either by gravity or other forces and collected in a collection bin (70) in preparation for packing. Alternately, the material could be feed directly in to a packing machine. The remaining material is coiled on a post-cut reel (67).

### Use

In use the patient selects an appropriate wrist support 30. A prefabricated wrist support 30 is selected, or the user can simply choose the assembly version and cut it to the appropriate shape. The cover layer is removed from the wrist support, then the wrist portion is engaged about the wrist with pressure applied to ensure that the adhesive is securely engaged on the wrist. The second portion is then applied against the back of the hand under pressure. The high shear strength of the adhesive along with the inelastic, high tensile strength support layer will brace the wrist from flexing downward over the carpal tunnel. The user may still flex the wrist upward, as well as side to side rotation. If further limitation against downward rotation is desired, the device can be preloaded by rotating the wrist upward prior to application of the second portion to the back of the hand. The wrist is thus supported without compression on the blood vessels, nerves, tendons, ligaments, etc. that are being treated.

The device can be easily removed when it is no longer needed. The user simply places the support device beneath hot running water that is at a temperature around 105-125 degrees Fahrenheit. Alternatively the user applies heat from a hair dryer to aid in removal of the support device. This temperature will not cause discomfort or harm to the user, but will release the adhesive, that is cause the adhesive to lose most of it's tack. The device can then simply be pulled off without discomfort and discarded.

An advantage of the disclosed invention is that it may be universally shaped for application to either wrist (see FIG. 10 or FIG. 11). The disclosed invention is lightweight, comfortable, not bulky and can be discretely worn, thus increasing compliance with its use. The disclosed device is easily applied, easy to transport in a small handbag or purse, has a low cost of manufacture, and can be discarded after a single use, thus not requiring cleaning. The disclosed invention utilizes the adhesive bonding of non-rigid material and human tissue to provide support and limit flexation of the wrist, without the need for or disadvantages of elastic and/or rigid materials. The heat releasable adhesive provides a means of removal with out painfully pulling on the hair or skin, thus improving practicality and user compliance. These devices could be shaped for use on other parts of the human by by way of example knees, ankles, and elbows.

### Treatment of Repetitive Stress Injuries

A preferred embodiment of the present invention is used to treat repetitive stress injuries such as Carpal Tunnel Syndrome as well as used to prevent these injuries. A suitable support is selected and cut to the appropriate size and shape. For example, a wrist support may be selected and trimmed to fit around all or a portion of the wrist to be treated with the extended portion trimmed to fit the back of the wrist. The cover layer is then pulled from the wrist support to expose the adhesive layer. The wrist support portion is applied directly onto the wrist so that the adhesive layer securely engages the support to the wrist. The wrist is then flexed to the appropriate position so that the appropriate preload or tension will be created on the wrist. The extended portion is then applied onto the wrist so the adhesive secures the wrist in the selected position.

The user is then unable to flex the wrist forward beyond this position without undue force and resulting pain. The user is then unable to flex the wrist forward beyond this position without undue force and resulting pain. This prevents further aggravation of the condition and prevents further damage. The wrist support is then easily removed when desired by applying heat to the wrist support, such as by soaking in hot water or applying heat from a hair dryer.

The foregoing disclosure and description of the invention is illustrative and explanatory thereof. Various changes in the details of the illustrated construction and methods may be made within the scope of the appended claims without departing from the spirit of the invention. No limitations are intended to the details of construction or design, herein shown, or to the methods described herein, other than are described in the claims below. The present invention should only be limited by the following claims and their legal equivalents.

1. A support device for treatment of carpal tunnel syndrome and ailments of the wrist, said device comprising:
a stretch-resistant support layer;
a first portion on said stretch-resistant support layer for engagement about the wrist of a patient;
a second portion extending from said first portion so that as said first portion is secured to the wrist, said second portion extends up the back of the hand; and
an adhesive layer on said stretch-resistant support layer to secure said first portion about the wrist of a patient and said second portion to the back of the hand; and
said adhesive layer having an adhesion strength which decreases in adhesion by at least 20% when its temperature is increased from 70 deg F. to a steady state temperature of 110 deg F. whereby minimizing discomfort to the user during removal by the application of heat.

2. The support device as described in claim 1, further wherein said stretch resistant member is a woven fabric with substantially equal elongation characteristics in both the weave and weft directions.

3. The support device as described in claim 2, further wherein said stretch resistant support layer has an ultimate yield strength of at least 25 N/cm.

4. The support device as described in claim 1, further wherein said stretch resistant support layer has an elongation of less than 15% at a load of 10 N/cm.

5. The support device as described in claim 1, further wherein said device generally forms the shape of a T.

6. The support device as described in claim 1, further wherein said device generally forms the shape of an L.

7. A disposable orthotic device for adhesive application to the body said device comprising:
a stretch-resistant support layer, with an ultimate yield strength of at least 25 N/cm and an elongation of less than 15% at a load of 10 N/cm,
an adhesive layer on said stretch-resistant member to secure said member to the body, said adhesive layer having a temperature sensitivity wherein said adhesion strength decreases in adhesion when its temperature is increased from 70 deg F. to a steady state temperature of less than 125 deg F. whereby minimizing discomfort to the user during removal by the application of heat.

8. The support device as described in claim 1, further wherein said stretch resistant support layer has substantially equal elongation characteristics in both the weave and weft directions/

9. The support device as described in claim 2, wherein said stretch-resistant support layer includes a high permeability uniformly woven fabric with a thickness of less than 15 mils.

10. The support device as described in claim 1, further wherein said stretch resistant support layer includes woven synthetic microfiber.

11. The support device as described in claim 1, further wherein said device generally forms the shape of a T.

12. The support device as described in claim 1, further wherein said device generally forms the shape of an L, contacting at least three sides of the wrist.

13. The support device as described in claim 1, shaped for ease of conformance to the wrist of a user.
